(19) United States
(12) Patent Application Publication
(10) Pub. No.: US 2026/0083763 A1
(43) Pub. Date: Mar. 26, 2026

(54) LIQUID COMPOSITIONS COMPRISING FIBER

(71) Applicant: Kenvue Brands LLC, Summit, NJ (US)

(72) Inventors: Katherine C. Dectis, Langhorne, PA (US); Nancy Edge, Summit, NJ (US); Patricia L. Golas, Somerville, NJ (US); Jian Guo Dong, Newtown Square, PA (US); Ibukunoluwa Adesuwa Oluwawemitan, Bronx, NY (US)

(21) Appl. No.: 19/339,624

(22) Filed: Sep. 25, 2025

Related U.S. Application Data
(60) Provisional application No. 63/699,340, filed on Sep. 26, 2024.

(51) Int. Cl. A61K 31/702; A61K 9/00; A61K 9/08; A61K 31/718; A61K 47/12

(52) U.S. Cl. CPC A61K 31/702; A61K 9/0095; A61K 9/08; A61K 31/718; A61K 47/12

(57) ABSTRACT

Liquid compositions comprising one or more therapeutic ingredients including fiber and processes of manufacturing the same. Such compositions can be used as a prebiotic dietary supplement to maintain and/or improve the gut microbiome and overall gut health of a human.

### CROSS-REFERENCE TO RELATED APPLICATIONS

This application claims the benefit of U.S. provisional application 63/699,340 filed on Sep. 26, 2024, the complete disclosure of which is hereby incorporated herein by reference for all purposes.

### 1. FIELD

The present disclosure relates to liquid compositions, and in particular liquid compositions comprising one or more therapeutic ingredients including fiber, and processes of manufacturing the same. The present disclosure further relates to the use of such liquid compositions as a prebiotic dietary supplement to maintain and/or improve the gut microbiome and overall gut health of a human.

### 2. BACKGROUND

The human gut microbiome has a role in overall health in influencing digestion, metabolism, and immune function. Prebiotics are among the various components that support gut health, for example, by fostering growth or activity of beneficial microorganisms such as certain bacteria and fungi while suppressing the growth of pathogenic bacteria. Prebiotics include inulin, fructooligosaccharides (FOS), galactooligosaccharides (GOS), xylooligosaccharides (XOS), and resistant starch. Despite the recognized benefits of prebiotics, there are several challenges associated with their use. For example, the stability and bioavailability of prebiotics in various dietary supplements remain important factors for effective formulations. Fibers such as inulin and FOS have been shown to have pasteurization processing challenges in acidic liquid matrices, where hydrolysis of the fibers can occur, degrading the fiber to sugars. Vega and Hansen (2015); Huebner et al. (2007). Additionally, during long term storage, hydrolysis of fibers such as FOS and XOS can occur in low pH (acidic) solutions. Courtin et al. (2009). Additionally, prebiotic efficacy typically requires relatively high dosages of several grams per serving which can pose solubility challenges in syrup products. Prebiotic fibers dissolved in a syrup also tend to increase water activity, which can exacerbate challenges with maintaining preservation and microbial robustness.

Orally administered dietary supplements can be administered in many forms, including solid forms, such as capsules, caplets, or tablets, and liquid forms, such as, solutions, e.g., syrups and elixirs, emulsions, or suspensions. Children and others may have trouble swallowing solid forms, e.g., whole tablets and even capsules. Therefore, it is desirable to provide dietary supplements either in a chewable solid form or a liquid form. For many, including the pediatric and geriatric populations, a liquid oral dosage form is preferable over chewable dosage form because of the ready swallowability without chewing of the liquid dosage form.

There remains a continued need in the art for novel prebiotic formulations, particularly in liquid solution form, that enhance stability, improve bioavailability, and maximize the beneficial effects of prebiotics on the gut microbiota. The present disclosure addresses these and other needs.

### 3. SUMMARY

The present disclosure provides liquid compositions including a therapeutic ingredient in an amount of from about 60% to about 85% by weight, based on the total weight of the liquid composition; and one or more sweeteners in an amount of from about 5% to about 35% by weight, based on the total weight of the liquid composition. The therapeutic ingredient includes dietary fiber, and the one or more sweeteners includes agave syrup.

In certain embodiments, the liquid composition can have a pH of from about 2 to about 6. In certain embodiments, the liquid composition can have a pH of from about 2.8 to about 4 or from about 4 to about 6.

In certain embodiments, the dietary fiber has an average degree of polymerization of less than 6. In particular embodiments, the dietary fiber can be a short chain fructooligosaccharides (scFOS).

In certain embodiments, the dietary fiber can have an average degree of polymerization of greater than or equal to 6. In particular embodiments, the dietary fiber can include resistant dextrin, resistant maltodextrin, or combinations thereof.

In certain embodiments, the liquid composition can further include one or more preservatives. The one or more preservatives can include potassium sorbate, sodium benzoate, or combinations thereof.

In certain embodiments, the dietary fiber can include resistant dextrin or resistant maltodextrin, and the liquid composition can remain stable for at least 3 months at 40° C./75% RH.

The present disclosure provides liquid compositions including a therapeutic ingredient in an amount of at least about 60% by weight, based on the total weight of the liquid composition; and one or more sweeteners in an amount of at least about 10% by weight, based on the total weight of the liquid composition. The therapeutic ingredient includes dietary fiber, and the one or more sweeteners is agave syrup.

In certain embodiments, the liquid composition can have a pH of from about 2 to about 6.

In certain embodiments, the dietary fiber can have an average degree of polymerization of greater than or equal to 6.

In certain embodiments, the liquid composition can remain stable for at least 3 months at 40° C./75% RH.

In certain embodiments, the dietary fiber can include resistant dextrin, resistant maltodextrin, or combinations thereof.

The present disclosure further provides methods of maintaining and/or improving the gut microbiome and overall gut health of a human, comprising administering the liquid compositions of the present disclosure to the human.

### 4. DETAILED DESCRIPTION

The presently disclosed subject matter relates to liquid compositions comprising one or more therapeutic ingredients including fiber and methods of manufacturing the same. Such compositions can be used as a prebiotic dietary supplement, for example, to maintain and/or improve the gut microbiome and overall gut health of a human. The incorporation of a soluble liquid fiber into an agave base to create a liquid fiber syrup in accordance with the present disclosure advantageously provides a convenient, easy to administer format of fiber supplementation for benefiting the digestive health of a human. Commercially available fiber supplements, in particular some powder formats, have been known for gritty textures and unpleasant tastes which can result in poor compliance and a negative consumer experience. Gummy formats also exist but are limited to children of older age that can safely consume the product as its youngest consumers, since gummies present a choking hazard for very young children. The present disclosure advantageously provides a liquid format of fiber supplementation that provides a high concentration of fiber per dose, a sufficient quantity of fiber for benefiting gut health at low dose (e.g., as low as 5 mL or 10 mL per day). Such liquid formats of the present disclosure can be administered to even very young children (e.g., as young as age 1) with a low dose needed per day.

These and other aspects of the disclosed subject matter are discussed in more detail below and in the Examples. For clarity, and not by way of limitation, this detailed description is divided into the following sub-portions:
4.1. Definitions;
4.2. Liquid Compositions;
4.3. Methods of Making Liquid Compositions;
4.4. Methods of Using Liquid Compositions; and
4.5. Features of Liquid Compositions.

### 4.1. Definitions

The terms used in this specification generally have their ordinary meanings in the art within the context of this disclosure and in specific context where each term is used. Certain terms are discussed below, or elsewhere in the specification, to provide additional guidance in describing the compositions and methods of the disclosure and how to make and use them.

As used in the specification and the appended claims, the singular forms “a,” “an,” and “the” include the plural referents unless the context clearly dictates otherwise. Thus, for example, reference to “an ingredient” includes mixtures of ingredients.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. The general convention in the scientific and technical literature is applied: the last decimal place of a numerical value indicates its degree of accuracy. Where no other error margins are given, the maximum margin is ascertained by applying the rounding-off convention to the last decimal place, for example for a measurement of 3.5%, the error margin is 3.45-3.54.

As used herein, “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The expression “one or more” is synonymous with “at least one” and includes individual components as well as mixtures/combinations.

All percentages, parts and ratios herein are based upon the total weight of the cleaning compositions of the present disclosure, unless otherwise indicated.

As used herein, the terms “% w/w” or “weight percent” refers to the percentage of an ingredient(s)/the total percentage by weight of the composition (100%). The terms “% w/w” or “weight percent” refer to the quantity by weight of a constituent or component. The terms “weight percent”, “wt-%”, “wt. %”, and “wt %” are used interchangeably.

All publications and patent applications cited in this specification are herein incorporated by reference, and for any and all purposes, as if each individual publication or patent application were specifically and individually indicated to be incorporated by reference. The present disclosure controls if there an inconsistency between the present disclosure and any incorporated publications or patents.

### 4.2. Liquid Compositions

Liquid compositions of the present disclosure include one or more therapeutic ingredients including fiber. In certain embodiments, the liquid compositions can include one or more therapeutic ingredients including fiber, one or more sweeteners, one or more flavoring agents, one or more pH adjusting agents, one or more preservatives, one or more bulk diluents, one or more additional additives, or combinations thereof.

### 4.2.1. Therapeutic Ingredients

The liquid composition can include one or more therapeutic ingredients. The one or more therapeutic ingredients can include fiber. In certain embodiments, the one or more therapeutic ingredients can include dietary fiber such as low molecular weight short chain fructooligosaccharides (scFOS) (e.g., Nutraflora® L95-S (Ingredion Corporation)) and higher molecular weight resistant dextrins or resistant maltodextrin (e.g., Fibersol® (ADM/Matsutani LLC), FiberSMART® (Anderson Advanced Ingredients), or Promitor® (Tate & Lyle)). In certain embodiments, the one or more therapeutic ingredients includes higher molecular weight resistant dextrins or resistant maltodextrin.

In certain embodiments, the one or more therapeutic ingredients can include fibers with an average degree of polymerization of less than 6, for example scFOS such as Nutraflora® L95-S (Ingredion Corporation); or can include fibers with an average degree of polymerization greater than or equal to 6, for example, resistant dextrins such as Fibersol® (ADM/Matsutani LLC), FiberSMART® (Anderson Advanced Ingredients), or Promitor® (Tate & Lyle) and other soluble corn fibers. Degree of polymerization (DP) is a fundamental parameter in polymer science that represents the average number of repeating monomer units within a polymer chain. It is calculated as the ratio of a polymer's molecular weight to the molecular weight of its repeating unit (monomer).

The one or more therapeutic ingredients can be present in an amount of from about 60% to about 90%, about 70% to about 85%, or about 75% to about 85% by weight, based on the total weight of the liquid composition. In certain embodiments, the one or more therapeutic ingredients can be present an amount of about 60%, about 70%, about 75%, about 80%, or about 85% by weight, based on the total weight of the liquid composition. In particular embodiments, the one or more therapeutic ingredients can be present in an amount of at least about 60%, at least about 65%, at least about 70%, at least about 80%, or at least about 81% by weight, based on the total weight of the liquid composition.

### 4.2.2. Sweeteners

The liquid composition can include one or more sweeteners. The one or more sweeteners can include agave syrup, organic agave syrup, glycerin, honey, monk fruit extract, stevia extract, Rebaudioside M (RebM), Rebaudioside A (RebA), or combinations thereof. In certain embodiments, the one or more sweeteners includes agave syrup or organic agave syrup.

The one or more sweeteners can be present in an amount of from about 0.005% to about 35%, about 10% to about 22%, or about 12% to about 20% by weight, based on the total weight of the liquid composition. In certain embodiments, the one or more sweeteners can be present in an amount of about 10%, about 13%, about 14%, about 15%, about 17%, about 19%, about 20%, about 25%, about 30%, about 35% by weight, based on the total weight of the liquid composition. In particular embodiments, the one or more sweeteners can be present in an amount of about 35%, about 30%, about 25% or less, about 20% or less, about 19% or less, about 17% or less, about 15% or less, about 13% or less, about 10% or less, or about 5% or less by weight, based on the total weight of the liquid composition. In certain embodiments, the one or more sweeteners can be present in an amount of at least about 10%, at least about 12%, at least about 14%, at least about 19%, at least about 20%, at least about 25%, or at least about 30% by weight based on the total weight of the liquid composition.

### 4.2.3. Flavoring Agents

The liquid composition can include one or more flavoring agents. A person skilled in the art will appreciate a wide variety of flavoring agents are suitable for use with the present disclosure.

The one or more flavoring agents can be present in an amount from about 0.01% to about 0.5%, about 0.1% to about 0.4%, or about 0.2% to about 0.3% by weight, based on the total weight of the liquid composition. In certain embodiments, the one or more flavoring agents can be present in an amount of about 0.01%, about 0.1%, about 0.2%, about 0.25%, about 0.3%, about 0.4%, or about 0.5% by weight, based on the total weight of the liquid composition. In particular embodiments, the one or more flavoring agents can be present in an amount of about 0.5% or less, about 0.4% or less, about 0.3% or less, about 0.25% or less, about 0.2% or less, or about 0.1% or less by weight, based on the total weight of the liquid composition.

### 4.2.4. pH Adjusting Agents

The liquid composition can include one or more pH adjusting agents. A person skilled in the art will appreciate a wide variety of pH adjusting agents are suitable for use with the present disclosure. In certain embodiments, for example and not by way of limitation, the one or more pH adjusting agents can include citric acid, malic acid, tartaric acid, oxalic acid, fumaric acid, lactic acid, vinegar, or combinations thereof. In particular embodiments, the one or more pH adjusting agents can include citric acid.

The one or more pH adjusting agents can be present in an amount of from about 0.005% to about 1%, about 0.05% to about 0.5%, about 0.05% to about 0.3%, or about 0.1% to about 0.2% by weight, based on the total weight of the liquid composition. In certain embodiments, the one or more pH adjusting agents can be present in an amount of about 0.005%, about 0.008%, about 0.01%, about 0.033%, about 0.05%, about 0.066%, about 0.1%, about 0.2%, about 0.3%, or about 0.4% by weight, based on the total weight of the liquid composition. In particular embodiments, the one or more preservatives can be present in an amount of about 1% or less, about 0.4% or less, about 0.3% or less, about 0.2% or less, about 0.15% or less, or about 0.1% or less by weight, based on the total weight of the liquid composition.

### 4.2.5. Preservatives

The liquid composition can include one or more preservatives. The one or more preservatives can include potassium sorbate, sodium benzoate, or combinations thereof. In certain embodiments, the one or more preservatives can include sodium benzoate. In certain embodiments, the one or more preservatives can include potassium sorbate.

The one or more preservatives can be present in an amount of from about 0.01% to about 5%, about 0.05% to about 4%, or about 0.1% to about 3% by weight, based on the total weight of the liquid composition. In certain embodiments, the one or more preservatives can be present in an amount of about 0.01%, about 0.1%, about 0.2%, or about 1% by weight, based on the total weight of the liquid composition. In particular embodiments, the one or more preservatives can be present in an amount of about 4% or less, about 3% or less, about 2% or less, about 1% or less, about 0.5% or less, about 0.3% or less, or about 0.1% or less by weight, based on the total weight of the liquid composition. For potassium sorbate, the preferred pH range is 4.5-4.9. This range also can correlate to pH 4.3-4.7 diluted 1:1 with reverse osmosis (RO) or (deionized) DI water, for ease of measurement due to the viscous nature of the solution. Sodium benzoate has a preferred pH range of <4.

### 4.2.6. Bulk Diluents

The liquid composition can include one or more bulk diluents. A person skilled in the art will appreciate a wide variety of bulk diluents are suitable for use with the present disclosure. In certain embodiments, the one or more bulk diluents can include water.

The one or more bulk diluents can be present in an amount of from about 0.1% to about 10%, about 1% to about 8%, or about 1% to about 5% by weight, based on the total weight of the liquid composition. In certain embodiments, the one or more bulk diluents can be present in an amount of about 1%, about 4%, about 5%, or about 8% by weight, based on the total weight of the liquid composition. In particular embodiments, the one or more bulk diluents can be present in an amount of about 10% or less, about 8% or less, about 6% or less, about 5% or less, or about 1% or less by weight, based on the total weight of the liquid composition.

### 4.2.7. Additional Additives

The liquid composition can include one or more additional additives. A person skilled in the art will appreciate a wide variety of additives are suitable for use with the present disclosure. In certain embodiments, the one or more additional additives can include one or more vitamins, one or more minerals, one or more botanical extracts, or combinations thereof.

### 4.2.8. Formulations

Liquid compositions of the present disclosure can include one or more therapeutic ingredients, one or more sweeteners, one or more flavoring agents, one or more pH adjusting agents, one or more preservatives, and one or more bulk diluents.

In certain embodiments, the liquid composition can include from about 60% to about 90% of one or more therapeutic ingredients; from about 5% to about 35% of one or more sweeteners; from about 0.01% to about 0.5% of one or more flavoring agents; from about 0.005% to about 1% of one or more pH adjusting agents; from about 0.01% to about 5% of one or more preservatives; and from about 0.1% to about 10% of one or more bulk diluents by weight, based on the total weight of the liquid composition.

In certain embodiments, the one or more therapeutic ingredients can include dietary fiber (short chain fructooligosaccharides); the one or more sweeteners can include organic agave syrup; the one or more pH adjusting agents can include citric acid; the one or more preservatives can include potassium sorbate; and the one or more bulk diluents can include water.

In certain embodiments, the one or more therapeutic ingredients can include dietary fiber (short chain fructooligosaccharides); the one or more sweeteners can include organic agave syrup; the one or more pH adjusting agents can include citric acid; the one or more preservatives can include sodium benzoate; and the one or more bulk diluents can include water.

In certain embodiments, the one or more therapeutic ingredients can include dietary fiber (resistant maltodextrin); the one or more sweeteners can include organic agave syrup; the one or more pH adjusting agents can include citric acid; the one or more preservatives can include sodium benzoate; and the one or more bulk diluents can include water.

### 4.3. Methods of Making Liquid Compositions

In certain embodiments, a method of manufacturing liquid compositions of the present disclosure is provided. In certain embodiments, the one or more therapeutic ingredients comprising fiber can be added to a tared vessel with a high shear mixer. The one or more bulk diluents and the one or more sweeteners can be added to the one or more therapeutic ingredients comprising fiber to form a first solution. The first solution can be mixed at 80% of the speed of the high shear mixer to form a second solution. The one or more preservatives, the one or more bulk diluents (e.g., water), and the one or more flavorings can be added to the second solution and mixed for at least 10 minutes to form a third solution. If needed, the pH of the third solution can be adjusted with the one or more pH adjusters to form a fourth solution. The resulting solution can then be transferred into sanitary vessels and passed through a pasteurizer, for example, at a temperature of between about 162° F. to about 175° F. prior to transferring into suitable containers such as bottles.

Methods of manufacturing liquid compositions of the present disclosure further provide, in certain embodiments, the one or more bulk diluents can be added to a vessel to form a first solution. The one or more preservatives can be added to the first solution to form a second solution. The second solution can be mixed (e.g., with an immersion blender until all materials are dissolved) to form a third solution. The third solution can be added to a mixing tank with side sweeps at a target mixing speed (about 800 RPM). A compound tank can be heated with water to a maximum of about 90° F. The one or more flavoring agents, the one or more sweeteners, and the one or more therapeutic agents with fiber can be added to the mixing tank in sequence to form a fourth solution. A circulation pump can be used (at about 30 hz) and the fourth solution can be mixed for at least about 10 minutes. The fourth solution can be adjusted for pH with the one or more pH adjusters to form a fifth solution. The fifth solution can be sent through a pasteurizer at a temperature between about 162° F. to about 175° F.

Methods of manufacturing liquid compositions of the present disclosure further provide, in certain embodiments, adding the one or more therapeutic ingredients with fiber to a tared vessel to form a first solution. The one or more diluents and the one or more sweeteners can be added in sequence to the first solution and mixed (e.g., at about 100 RPM) to form a second solution. The one or more preservatives, the one or more flavoring agents, and the one or more diluents can be added in sequence to the second solution and mixed (e.g., for at least about 10 minutes) to form a third solution. The pH of the third solution can be adjusted with the one or more pH adjusters to form a fourth solution. The fourth solution can be heated (e.g., to approximately 160° F. to 170° F.) and then transferred into product packaging such as bottles.

### 4.4. Methods of Using Liquid Compositions

Liquid compositions of the present disclosure can be used alone. Methods of using the liquid compositions as a prebiotic dietary supplement to maintain and/or improve the gut microbiome and overall gut health of a human are provided. Such methods can include administering liquid compositions of the present disclosure to a human.

### 4.5. Features of Liquid Compositions

pH. The present disclosure provides liquid compositions including one or more therapeutic ingredients with fiber. Such compositions can have a pH of from about 2 to about 6, about 2.5 to about 4, or about 2.8 to about 4. In certain embodiments, liquid compositions including one or more therapeutic ingredients with fiber having an average degree of polymerization of less than 6 can have a pH of from about 4 to about 6, about 4.5 to about 5, or about 4.5 to about 4.9. In certain embodiments, such liquid compositions can have a pH of about 4, about 4.5, about 4.6, about 4.7, about 4.8, or about 5. In certain embodiments, liquid compositions including one or more therapeutic ingredients with fiber having an average degree of polymerization of greater than or equal to 6 can have a pH in the range of from about 2.8 to about 4, about 2.9 to about 3.5, or about 3 to about 3.5. In certain embodiments, such formulations can have a pH of 4 or less, 3.5 or less, 3 or less, or 2.8 or less.

Density. The density of liquid compositions including one or more therapeutic ingredients with fiber having an average degree of polymerization of less than 6 can range from about 1.1 g/mL to about 1.5 g/mL, about 1.2 g/mL to about 1.4 g/mL, or about 1.287 g/mL to about 1.437 g/mL. The density of liquid compositions including one or more therapeutic ingredients with fiber having an average degree of polymerization of greater than or equal to 6 can be from about 1.1 g/mL to about 1.5 g/mL, or about 1.2 g/mL to about 1.4 g/mL.

Percentage of Dissolved Solids (Brix Measurement). The brix measurement of liquid compositions including one or more therapeutic ingredients with fiber having an average degree of polymerization of less than 6 can range from about 55% to about 80%, about 60% to about 75%, or about 61.7% to about 75.4%. The brix measurement of liquid compositions including or more therapeutic ingredients with fiber having an average degree of polymerization of greater than or equal to 6 can be from about 65% to about 80%, about 65% to about 75%, or about 67% to about 75%.

Molecular Weight. Liquid compositions including one or more therapeutic ingredients with fiber having an average degree of polymerization of greater than or equal to 6 can have a molecular weight of about 2,000 with an average degree of polymerization of 11.

### 5. EXAMPLES

The following Examples are intended to illustrate, but not to limit, the disclosed subject matter in any manner, shape, or form, either explicitly or implicitly.

### Example 1: FOS Fiber Syrups with Potassium Sorbate

The present example was prepared using the formula in Table 1 as Formulation 1.

TABLE 1
Formulation 1

| | | Amount | |
|---|---|---|---|
| | | mg/Serving | |
| Ingredient | Function | (5 mL) | % W/W |
| Dietary Fiber (Short | Therapeutic | 5542.87 | 81.39 |
| Chain | Ingredient | | |
| Fructooligiosaccharides)¹ | | | |
| Organic Agave Syrup | Bulk Sweetener | 1170.39 | 17.19 |
| Flavor 1 | Flavor | 15.49 | 0.23 |
| Citric Acid | pH adjusting agent | 6.81 | 0.10 |
| Potassium Sorbate | Preservative | 6.81 | 0.10 |
| Water | Bulk Diluent | 67.56 | 0.99 |
| TOTAL | | 6810.0² | 100.0 |

¹Commercially available as Nutraflora ® L95-S from the Ingredion Corporation (minimum 66.5% by weight fiber; 27-30% by weight water)
²a 10 mL serving or dose is administered as 13620.00 mg

### Processing Directions (Formulation 1):

1. Water was added to a stainless-steel vessel.
2. Potassium Sorbate was added to the vessel of Step 1.
3. The solution from Step 2 was mixed with an immersion blender until all powders were dissolved.
4. The contents of the vessel of Step 3 were added to a mixing tank.
5. The mixing tank side sweeps were started. The mixing was continued at a target mixing speed (about 800 rpm) for the side sweeps.
6. The compound tank was heated with a water maximum of 90° F.
7. Flavor 1, Organic Agave Syrup, and Dietary Fiber (Short Chain Fructooligosaccharides; Nutraflora® L-95S) were added to the mixing tank in sequence.
8. Once all the Organic Agave Syrup and Dietary Fiber (Short Chain Fructooligosaccharides; Nutraflora® L-95S) were added, the circulation pump was turned on to a target speed of about 30 hz. The solution was mixed at the target settings for a mixing time of not less than about 10 minutes.
9. A sample was pulled from the solution and its pH was measured. 33% of Citric Acid was added to the mixing tank.
10. Jet shear was turned on to about 1,200 RPM to about 2,600 RPM (Target: about 1,400 RPM).
11. The solution was mixed for no less than about 20 minutes with heat.
12. Another sample was pulled and its pH was measured. The target pH was 4.5. If the sample pH was >4.6, moved to Step 13. If sample's pH was 4.4-4.6, moved to Step 17.
13. Another 33% of Citric Acid was added to the mixing tank. The solution was mixed and recirculated for no less than about 10 minutes with heat.
14. Another sample was pulled and its pH measured. If sample's pH was >4.6, moved to Step
15. If sample's pH was 4.4-4.6, moved to Step 17.
15. Another 33% of Citric Acid was added to mixing tank. The solution was mixed and recirculated for no less than about 10 minutes with heat.
16. Another sample was pulled and its pH measured. A pH within the range of 4.4-4.6 was confirmed and recorded.
17. The solution was transferred to a holding tank.
18. The solution was sent through a pasteurizer at a temperature between about 162° F. to about 175° F., through the in-line 10 mesh screen, to the filler.
19. The minimum filler bowl temperature (145° F.) was set and held in the filler bowl. If the filler bowl temperature with the solution dropped below 145° F., the solution was diverted back to the pasteurizer.

### Example 2: FOS Fiber Syrups with Sodium Benzoate

The present example was prepared using the formula in Table 2 as Formulation 2.

TABLE 2
Formulation 2

| | | Amount | |
|---|---|---|---|
| | | mg/Serving | |
| Ingredient | Function | (5 mL) | % W/W |
| Dietary Fiber (Short | Therapeutic | 5473.68 | 81.39 |
| Chain | Ingredient | | |
| Fructooligiosaccharides)¹ | | | |
| Organic | Bulk | 903.21 | 13.43 |
| Agave Syrup | Sweetener | | |
| Flavor 1 | Flavor | 14.76 | 0.22 |
| Citric | pH adjusting | 17.22 | 0.26 |
| Acid | agent | | |
| Sodium Benzoate | Preservative | 6.73 | 0.10 |
| Water | Bulk Diluent | 309.33 | 4.60 |
| TOTAL | | 6725.0² | 100.0 |

¹Commercially available as Nutraflora ® L95-S from the Ingredion Corporation
²a 10 mL serving or dose is administered as 13450.0 mg

### Processing Directions (Formulation 2):

1. To a tared vessel equipped with high shear mixer, the Dietary Fiber (Short Chain Fructooligosaccharides; Nutraflora® L-95S) was added to achieve suitable volume for mixing.
2. Water and Organic Agave Syrup were added in sequence to the vessel in Step 1 and mixed at 80% of the speed of the high shear mixer.
3. Sodium Benzoate, Flavor 1, and Water were added in sequence to the vessel in Step 2 and mixed for not less than about 10 minutes.
4. The pH was adjusted (if necessary) to a pH of 4.0-4.3 with Citric Acid.
5. The solution was transferred to sanitary vessels and passed through a pasteurizer at a temperature between about 162° F. to about 175° F., prior to transfer into bottles.

### Example 3: Resistant Dextrin Fiber Syrups with Sodium Benzoate

The present example was prepared using the formula in Table 3 as Formulation 3.

TABLE 3
Formulation 3

| | | Amount | |
|---|---|---|---|
| | | mg/Serving | |
| Ingredient | Function | (5 mL) | % W/W |
| Dietary | Therapeutic | 5406.75 | 80.10 |
| Fiber (Resistant | Ingredient | | |
| Dextrin)¹ | | | |
| Agave Syrup | Bulk Sweetener | 961.00 | 14.25 |
| Citric | pH adjusting | 28.00 | 0.40 |
| Acid | agent | | |
| Sodium Benzoate | Preservative | 6.75 | 0.1 |
| Flavor 1 | Flavor | 13.5 | 0.2 |
| Water | Bulk Diluent | 334.00 | 4.95 |
| Total | | 6750.0 | 100 |

¹Commercially available as Fiberso1 ® from ADM/Matsutani LLC (minimum 68.5% by weight fiber; 28.5-31.5% by weight water)

The present example was prepared using the formula in Table 5 as Formulation 5.

TABLE 4
Formulation 4

| | | Amount | |
|---|---|---|---|
| | | (mg)/Serving | |
| Ingredient | Function | (5 mL) | % W/W |
| Dietary | Therapeutic | 5062.5 | 75 |
| Fiber (Resistant | Ingredient | | |
| Dextrin)¹ | | | |
| Agave Syrup | Bulk Sweetener | 1336.5 | 19.8 |
| Citric | pH adjusting | 30.75 | 0.45 |
| Acid | agent | | |
| Sodium Benzoate | Preservative | 6.75 | 0.1 |
| Flavor 1 | Flavor | 20.25 | 0.3 |
| Water | Bulk Diluent | 293.25 | 4.35 |
| Total | | 6750 | 100 |

¹Commercially available as FiberSMART ® from Anderson Advanced Ingredients (minimum 71% by weight fiber; 27.6-29% by weight water)

TABLE 5
Formulation 5

| | | Amount | |
|---|---|---|---|
| | | mg/Serving | |
| Ingredient | Function | (5 mL) | % W/W |
| Dietary | Therapeutic | 4995 | 74 |
| Fiber (Resistant | Ingredient | | |
| Dextrin)¹ | | | |
| Agave Syrup | Bulk Sweetener | 1336.5 | 19.8 |
| Citric | pH adjusting | 33.75 | 0.5 |
| Acid | agent | | |
| Sodium Benzoate | Preservative | 6.75 | 0.1 |
| Flavor 1 | Flavor | 20.25 | 0.3 |
| Water | Bulk Diluent | 357.75 | 5.3 |
| Total | | 6750 | 100 |

¹Commercially available as Promitor ® from Tate & Lyle (minimum 63.9% by weight fiber; 27-29% by weight water)

### Processing Directions (Formulation 3, Formulation 4, and Formulation 5):

1. To a tared vessel, the Dietary Fiber (Resistant Maltodextrin) was added to achieve suitable volume for mixing.
2. Water and Organic Agave Syrup was added in sequence to the vessel in Step 1 and mixed at about 100 RPM.
3. Sodium Benzoate, Flavor 1, and Water were added in sequence to the vessel in Step 2 and mixed for not less than about 10 minutes.
4. The pH of the solution was adjusted to a pH of about 3 with Citric Acid.
5. The solution was heated on a hot plate to approximately 160° F. to 170° F. and then transferred to bottles.

### Example 4: Stability Data

The formulations shown in Examples 1-3 (Formulations 1-5) were stored in various environments and evaluated for stability. The amount of dietary fiber was analyzed at each time point in accordance with the methods provided below.

### Assay Methods for Total Dietary Fiber Content

Method for Analysis of Total Dietary Fiber for Short Chain Fructooligosaccharides (Nutraflora® L95-S) (Formulation 1 and Formulation 2). The following method was used to analyze the dietary fiber level. Fructans were first extracted with water. The extract was then centrifuged, filtered, and an appropriate dilution was injected for the analysis of free sugars. An aliquot of the filtrate was also subjected to treatment by enzymes to liberate fructose and glucose form the fructans. The total fructose and glucose content was determined by HPAEC (High-Performance Anion Exchange Chromatography). The fructose and glucose released from fructan was then calculated and used to determine the total fructan content. This method is described as the Official Method No. 997.08, Official Methods of Analysis of the Association of Official Agricultural Chemists (AOAC) International (modified), 17th Ed., AOAC International. The method is derived from the publication Stöber, P., Bénet, S., and Hischenhuber, C., “Simplified Enzymatic High-Performance Anion Exchange Chromatographic Determination of Total Fructans in Food and Pet Food-Limitations and Measurement Uncertainty,” Journal of Agricultural and Food Chemistry, 52(Modified)(8):2137-2146 (2004), incorporated herein by reference.

Method for Analysis of Total Dietary Fiber for Resistant Maltodextrin (Fibersol®) (Formulation 3). The following method was used to determine the dietary fiber level. Resistant Maltodextrin test portions were first gelatinized with alpha-amylase and digested with enzymes to break down starch and protein. For the measurement of high molecular weight dietary fiber (HMWDF), ethanol was added and the insoluble and high molecular weight soluble dietary fiber was filtered, washed, dried and weighed. A portion of the residue was analyzed for protein and for ash. The high molecular weight dietary fiber was calculated from the residue weight and corrected for protein, ash and process blanks. The low molecular weight soluble dietary fiber (LMWSDF) in the filtrate was concentrated, desalted through de-ashing cartridges and finally analyzed by size exclusion High-Performance Liquid Chromatography (HPLC) equipped with a refractive index detector (RI). The quantification of LMWSDF was based on the response factor of external glucose calibration standards against the internal standard Diethylene glycol. The response of glucose was assumed to be equivalent to the response of all the LMWSDF compounds when using RI as the detection platform. This method is referenced in the Official Methods of Analysis, Method 2001.03, Association of Official Agricultural Chemists (AOAC) International, Gaithersburg, MD, USA (Modified).

Method for Analysis of Total Dietary Fiber for Resistant Maltodextrin (FiberSMART®—Formulation 4) (Promitor®—Formulation 5). The Total Dietary Fiber (TDFM) analysis without separation of insoluble and high molecular weight soluble fiber Test portions were first enzymatically digested with pancreatic alpha-amylase and amyloglucosidase (AMG) for 16 hours at 37° C. in sealed 250 mL bottles in a shaking water bath while mixing with sufficient vigor to maintain continuous suspension. During this step, non-resistant starch was solubilized and hydrolyzed to glucose and maltose by the combined action of the two enzymes. The reaction was terminated by pH adjustment and heat. Protein in the sample was digested with protease. For the measurement of high molecular weight dietary fiber (HMWDF) ethanol was added and the digestate was filtered, washed, dried, and weighed. A portion of the residue was analyzed for protein and ash. The high molecular weight dietary fiber was calculated from the residue weights and corrected for protein, ash, and process blanks. The low molecular weight soluble dietary fiber (LMWSDF), also known as resistant oligosaccharides, in the filtrate was concentrated, desalted through de-ashing cartridges and finally analyzed by size exclusion High-Performance Liquid Chromatography (HPLC) equipped with a refractive index (RI) detector. This method is referenced in the Official Methods of Analysis, Methods 2009.01 and 2011.25, Association of Official Agricultural Chemists (AOAC) International (modified).

The results are shown in Tables 6 and 7 below.

TABLE 6
Stability Results for Storage at 40° C./75% RH

| | | Dietary | Dietary | Dietary | Dietary |
|---|---|---|---|---|---|
| | | Fiber | Fiber | Fiber | Fiber |
| | Dietary | Level | Level | Level | Level |
| | Fiber | (g/5 mL) | (g/5 mL) | (g/5 mL) | (g/5 mL) |
| | Level | 1 month, | 2 months, | 3 months, | 6 months, |
| | (g/5 mL) | 40° C./ | 40° C./ | 40° C./ | 40° C./ |
| Formulation | Initial | 75% RH | 75% RH | 75% RH | 75% RH |
| 1 | 3.67 | 3.6 | 3.41 | 2.99 | 2.54 |
| 2 | 3.5 | 2.67 | 2.28 | 1.61 | 0.68 |
| 3 | 3.8 | 3.78 | 3.81 | 3.44 | 3.24* |
| 4 | 3.45 | 3.14 | 3.34 | 3.14 | 2.94* |
| 5 | 3.83 | 3.63 | 3.65 | 3.59 | 3.36* |

RH = Relative Humidity
Note:
The minimum acceptable fiber content and label claim is 2.0 grams per 5 mL serving.
*extrapolated results to 6 months

TABLE 7
Stability Results for Storage at 50° C.

| | | Dietary | Dietary | Dietary |
|---|---|---|---|---|
| | Dietary | Fiber | Fiber | Fiber |
| | Fiber | Level | Level | Level |
| | Level | (g/5 mL) | (g/5 mL) | (g/5 mL) |
| | (g/5 mL) | 1 week, | 2 weeks, | 13 weeks, |
| Formulation | Initial | 50° C. | 50° C. | 50° C. |
| 1 | 3.6 | 3.47 | 3.49 | 2.86* |
| 2 | 3.08 | 2.55 | 2.23 | 0* |

*extrapolated results to 13 weeks

The stability results indicate that the formulations using short chain fructooligosaccharides (scFOS), Formulations 1 and 2, degraded significantly in both conditions. The formulation using sodium benzoate and a lower pH (Formulation 2) degraded at the highest rate in both storage conditions, while Formulation 1 met acceptance criteria (>2.0 grams of fiber per 5 mL serving).

### Example 5: Antimicrobial Effectiveness Testing (AET)

The formulations shown in Examples 1-3 (Formulations 1-5) were stored and USP <51> was performed which is the United States Pharmacopeia's Antimicrobial Effectiveness Test (AET), a standardized method to assess a product's ability to inhibit the growth of a panel of five specific microorganisms over a 28-day period. The method provided in USP-NF <51> Antimicrobial Effectiveness Testing, USPC, DOI: https://doi.org/10.31003/USPNF_M98790_03_01 (2025).

The USP <51> Pharmaceopia criteria for Category 3 Products (Oral products other than antacids, made with aqueous bases or vehicles) is provided in Table 8 below.

TABLE 8
USP <51> Pharmaceopia Criteria for Category 3 Products
USP <51> Pharmacopeia Criteria for Category 3 Products

| Class of | Criteria for Category 3 |
|---|---|
| microorganism | Products |
| Bacteria | Not less than 1.0 log10 |
| | reduction of the initial count |
| | in 14 days, and no increase in |
| | the count of 14 days in 28 |
| | days |
| Fungi (Yeast & Mold) | No increase in initial |
| | calculated count at 14 and 28 |
| | days |

The AET results related to Formulation 1 are provided in Tables 9, 10 and 11 below.

TABLE 9
AET results summary: sc-FOS Fiber Syrup Batch (Formulation 1)

| | (Initial/ | (T = 6 M |
|---|---|---|
| Storage Condition | T = 0) | 40° C./75% RH) |
| Potassium sorbate | 6.28 mg/ml | 2.52 mg/5 ml |
| concentration | (0.092%) | (0.037%) |
| Test Conclusion (USP <51>) | Passed | Passed |

TABLE 10
AET Results: scFOS Fiber Syrup Benchtop Batch (Formulation 1 -
varying preservative levels to simulate loss during shelf life)

| | | | | |
|---|---|---|---|---|
| Potassium | 0.09% | 0.07% | 0.05% | 0% |
| sorbate | | | | |
| concentration | | | | |
| Test Conclusion | Passed | Passed | Passed | Passed |
| (USP <51>) | | | | |

TABLE 11
AET Results: scFOS Fiber Syrup Benchtop at different
pH ranges with 0.1% potassium sorbate (Formulation 1)

| | | | |
|---|---|---|---|
| Potassium | 0.1% | 0.1% | 0.1% |
| sorbate | | | |
| concentration | | | |
| pH | 4.5 | 4.8 | 5.0 |
| Test | Passed | Passed | Passed |
| Conclusion | | | |
| (USP <51>) | | | |

The AET results for Formulation 3 are provided in Table 12 below.

TABLE 12
AET Results for Formulation 3 (Fibersol ®)

| | | T = | T = | T = |
|---|---|---|---|---|
| Timepoint & | | 1 Month, | 2 Month, | 3 Month, |
| Storage | T0/ | 40° C./ | 40° C./ | 40° C./ |
| Condition | Initial | 75% RH | 75% RH | 75% RH |
| Sodium | 0.1% | 0.1% | 0.1% | 0.07% |
| Benzoate | | | | |
| concentration | | | | |
| Test | Passed | Passed | Passed | Passed |
| Conclusion | | | | |
| (USP <51>) | | | | |

The AET results for Formulation 4 are provided in Table 13 below.

TABLE 13
AET results for Formulation 4 (FiberSMART ®)

| | | T = | T = | T = |
|---|---|---|---|---|
| Timepoint & | | 1 Month, | 2 Month, | 3 Month, |
| Storage | T0/ | 40° C./ | 40° C./ | 40° C./ |
| Condition | Initial | 75% RH | 75% RH | 75% RH |
| Sodium | 0.1% | 0.1% | 0.085% | 0.085% |
| Benzoate | | | | |
| concentration | | | | |
| Test | Passed | Passed | Passed | Passed |
| Conclusion | | | | |
| (USP <51>) | | | | |

The AET results for Formulation 5 are provided in Table 14 below.

TABLE 14
AET Results for Formulation 5 (Promitor ®)

| | | T = | T = | T = |
|---|---|---|---|---|
| Timepoint & | | 1 Month, | 2 Month, | 3 Month, |
| Storage | T0/ | 40° C./ | 40° C./ | 40° C./ |
| Condition | Initial | 75% RH | 75% RH | 75% RH |
| Sodium | 0.1% | 0.1% | 0.1% | 0.1% |
| Benzoate | | | | |
| concentration | | | | |
| Test | Passed | Passed | Passed | Passed |
| Conclusion | | | | |
| (USP <51>) | | | | |

### Example 6: Precipitation Observations; Stability Study Results; AET Study Results

For liquid formulations containing FOS and agave, an acidic agent can be needed in order to preserve the shelf life of the formulation for a dietary supplement product, especially if stored at room temperature. Acidity from citric acid or another acid can help prevent microbial growth, and a preservative system can also be used to help prolong shelf life and prevent additional microorganisms from growing in the formulation. The adjustment of the citric acid input to achieve the optimal pH range for both preservative efficacy and fiber stability was needed for such formulations.

Sodium benzoate as preservative and citric acid QS to achieve a pH of 3.8-4.3 resulted in the FOS fiber degrading to insufficient fiber levels (Tables 6 and 7—Formulation 2). FOS can degrade at low pH, but it can depend on the solids content and the specific matrix of the formula. A higher pH was needed in order to have more stable fiber content. However, sodium benzoate's efficacy decreases at around pH>4.3. Thus, a new preservative was assessed (potassium sorbate) which has been shown to have an improved efficacy at around pH<5.0. Challenges existed with the change in preservative, since high concentrations in a low-water content formulation at the current pH range, potassium sorbate can cause precipitation issues.

About 0.1% potassium sorbate showed no precipitation issues, where higher concentrations (0.2% and 0.3%) were not feasible. An accelerated heat study was conducted at pH 4.5 with 0.1% potassium sorbate, which was shown to achieve a more stable fiber content (Table 7—Formulation 1). The water content of the formulation was adjusted to yield a lower water activity of about 0.80-0.82 for enhanced microbiological stability by reducing added water from about 4.6% to about 1%, which did not result in any precipitation issues. Additional accelerated stability of this formulation was conducted that further supports the stability of the FOS fiber in the agave matrix at a higher pH (Table 6—Formulation 1). AET studies were also conducted to confirm that the preservative efficacy of the system was maintained at specific pH (4.5, 4.8, 5.0) (Table 11), all meeting USP <51> criteria, Table 8. AET was also conducted during an accelerated stability study, which met USP criteria (Table 9), as well as under conditions with reduced preservative levels, to simulate loss during shelf life, all meeting USP criteria (Table 10).

Liquid compositions containing short chain-FOS (scFOS) had challenges with the higher than optimal pH and overall fiber stability. New fiber sources were utilized to achieve a similar overall formulations (acidified, agave-based syrup) with a more stable fiber. Several fibers were screened on the benchtop at a 5 and 10 mL serving size, and resistant dextrins fibers were determined to have the best mixability and stability in the agave base. The fibers evaluated in the Examples above were Fibersol® from ADM, Promitor® from Tate & Lyle, and FiberSMART® from Anderson. It was challenging in this matrix to achieve a fiber syrup that is high in fiber content (2.8 g/5 mL serving), stable over time, and can maintain a low pH for microbial robustness. Resistant dextrin in the liquid formulations of the present disclosure was able to meet these desired metrics. Several resistant dextrins at pH 3 were put on an accelerated stability study (40° C. for 3 months) and were found to be highly stable over time having at least 2.8 g of fiber per 5 mL serving (Table 6—Formulations 3, 4, and 5). Anti-microbial effectiveness tests were also performed over the 3-month stability study and the resistant dextrin prototypes were found to be very effective in preventing microbial contamination (Tables 12, 13, and 14).

1. A liquid composition, comprising:
a therapeutic ingredient in an amount of from about 60% to about 85% by weight, based on the total weight of the liquid composition; and
one or more sweeteners in an amount of from about 5% to about 35% by weight, based on the total weight of the liquid composition;
wherein the therapeutic ingredient comprises dietary fiber, and the one or more sweeteners comprises agave syrup.

2. The liquid composition of claim 1, wherein the liquid composition has a pH of from about 2 to about 6.

3. The liquid composition of claim 2, wherein the liquid composition has a pH of from about 2.8 to about 4 or from about 4 to about 6.

4. The liquid composition of claim 1, wherein the dietary fiber has an average degree of polymerization of less than 6.

5. The liquid composition of claim 4, wherein the dietary fiber is a short chain fructooligosaccharides (scFOS).

6. The liquid composition of claim 1, wherein the dietary fiber has an average degree of polymerization of greater than or equal to 6.

7. The liquid composition of claim 6, wherein the dietary fiber comprises resistant dextrin, resistant maltodextrin, or combinations thereof.

8. The liquid composition of claim 1, further comprising one or more preservatives.

9. The liquid composition of claim 8, wherein the one or more preservatives comprises potassium sorbate, sodium benzoate, or combinations thereof.

10. The liquid composition of claim 1, wherein the dietary fiber comprises resistant dextrin or resistant maltodextrin, and wherein the liquid composition remains stable for at least 3 months at 40° C./75% RH.

11. A liquid composition, comprising:
a therapeutic ingredient comprising fiber in an amount of at least about 60% by weight, based on the total weight of the liquid composition; and
one or more sweeteners in an amount of at least about 10% by weight, based on the total weight of the liquid composition;
wherein the therapeutic ingredient comprises dietary fiber, and the one or more sweeteners consists of agave syrup.

12. The liquid composition of claim 1, wherein the liquid composition has a pH of from about 2 to about 6.

13. The liquid composition of claim 1, wherein the dietary fiber has an average degree of polymerization of greater than or equal to 6.

14. The liquid composition of claim 13, wherein the liquid composition remains stable for at least 3 months at 40° C./75% RH.

15. The liquid composition of claim 1, wherein the dietary fiber comprises resistant dextrin, resistant maltodextrin, or combinations thereof.

16. A method of maintaining and/or improving the gut microbiome and overall gut health of a human, comprising: administering the liquid composition of claim 1 to the human.
